(19) United States
(12) Patent Application Publication
(10) Pub. No.: US 2007/0196352 A1
(43) Pub. Date: Aug. 23, 2007

(54) Vegf-induced genes and their therapeutic use

(76) Inventors: Ian Zachary, London (GB); Dan Liu, London (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/572,126

(22) PCT Filed: Sep. 16, 2004
(86) PCT No.: PCT/GB04/03956

§ 371 Date: Jan. 16, 2007

(30) Foreign Application Priority Data
Sep. 16, 2003 (GB) 0321694.2

(51) Int. Cl. A61K 38/46; A61K 38/17; A61K 38/18

(52) U.S. Cl. 424/94.6; 514/12

(57) ABSTRACT

The present invention concerns products for therapeutic use comprising a gene, or product thereof, identified as upregulated by VEGF (vascular endothelial growth factor). Products of the invention can be used, for example, in arterioprotection or promoting angiogenesis.

### FIELD OF THE INVENTION

This invention relates to genes induced by VEGF, and their gene products, for which a new therapeutic use has been found.

### BACKGROUND OF THE INVENTION

Vascular endothelial growth factor (VEGF or VEGF-A) is essential for endothelial cell differentiation (vasculogenesis) and angiogenesis during development of the embryonic vasculature, and plays a major role in neovascularisation in a variety of disease states. Two protein tyrosine kinase receptors for VEGF, KDR/Flk-1 and Flt1, are essential for embryonic vascular development. The biological effects of VEGF in endothelia are mediated primarily via KDR whereas recent findings indicate that Flt-1 may act as a negative regulator of KDR. Neuropilin-1 is a non-tyrosine kinase receptor for VEGF which may function as a ‘docking’ co-receptor for KDR. After binding to KDR, VEGF activates multiple early signalling cascades in endothelial cells, including phospholipase C-γ leading to increased PKC activity and mobilization of intracellular Ca²⁺, activation of p42/p44 ERKs 1 and 2, phosphatidylinositol 3′-kinase-dependent Akt/PKB activity and tyrosine phosphorylation of p125 Focal Adhesion Kinase. Subsequently, VEGF elicits an array of biological activities in vivo and in vitro including the survival, proliferation and migration of endothelial cells, endothelial production of NO and PGI₂, and increased vascular permeability.

VEGF-induced upregulation of several genes has been reported in endothelial cells; see, for example, Hernandez et al, J Exp Med. 2001l, 193:607-20; Lee et al, J Biol Chem. 2002, 277:10445-51; Arkonac et al, J Biol Chem. 1998, 273:4400-5; Mason et al, Am J Physiol Cell Physiol. 2002 March, 282(3):C578-87; Kahn et al, Am J Pathol. 2000, 156:1887-900; and Bell et al, J Cell Sci. 2001 August, 114(Pt 15):2755-73. However, the programme of gene expression regulated by VEGF remains unclear. In particular, there is a lack of information regarding the early gene expression events and transcription factors that are likely to play a central role in signal transduction downstream of KDR, and in mediating the long-term biological responses induced by VEGF.

WO98/20027 describes the utility of VEGF in treating internal hyperplasia, inter alia, and a system for gene delivery.

### SUMMARY OF THE INVENTION

The present invention is based on a study intended to provide insights into the molecular mechanisms through which VEGF exerts its biological activities. In this study, the pattern of gene expression regulated by VEGF was examined, using analysis of oligonucleotide arrays representing more than 15,000 human genes, combined with real-time quantitative RT-PCR. The results show that, while some of the VEGF-regulated genes identified have previously been linked to angiogenesis, most have no previously established role in either angiogenesis or endothelial function. A notable feature of these findings is the rapid upregulation of several immediate early genes, including a family of closely-related orphan nuclear receptors. On the one hand, these findings should advance our understanding of the mechanisms underlying angiogenesis and other cellular functions regulated by VEGF. On the other, they indicate that a desirable therapeutic effect can be obtained by the use of genes that are upregulated by VEGF. In particular, genes that are upregulated by VEGF have therapeutic utility in the same sense as VEGF, as described, for example, in WO98/20027, and including arterioprotection and the promotion of angiogenesis. Further utilities lie in diagnosis and in screening for active drugs.

It will be appreciated that an appropriate gene may be expressed, in situ, and that the corresponding gene product may be used instead. Either may be administered in conventional manner.

### DESCRIPTION OF THE INVENTION

Any product described herein can be used in the same general manner as is described for VEGF in WO98/20027. Patients and conditions that can be treated will be apparent to one of ordinary skill in the art. Diagnostic tests and screening techniques utilising products described herein will also be readily understood and practiced by one of ordinary skill in the art.

For example, on the basis of the discoveries reported here, it will be apparent that certain genes and gene products can be used in therapy, e.g. of internal hyperplasia, leading to stenosis or restenosis, and hypertension. The gene or gene product (which for the purposes of this specification includes active fragments, as will be understood by those skilled in the art) may be administered directly to the site needing treatment, by known means, or periadventitiously, e.g. as described in detail in WO98/20027. Suitable vectors etc are also described there; the content of that document is incorporated herein by reference.

The Study reported below, i.e. oligonucleotide array analysis of oligonucleotide arrays representing approximately 18,000 human genes, identified several VEGF-regulated genes that have previously been reported to be either upregulated by VEGF and/or associated with angiogenesis. The largest number of genes identified in the present study are VEGF-regulated genes that have not previously been implicated in angiogenesis or endothelial cell biology. Comparison of the results obtained from oligonucleotide arrays with those of specific quantitative RT-PCR analysis supports the view that oligonucleotide arrays are a useful predictor of VEGF-regulated gene expression. Subsequent RT-PCR of induced or downregulated genes, demonstrated that 16 out of 23 selected genes identified from arrays were regulated by VEGF, and in each case both the pattern and the magnitude of change in gene expression detected in arrays and RT-PCR showed close agreement. The findings presented here represent the most comprehensive study of the regulation of gene expression by VEGF to date, previous studies having analysed smaller numbers of genes and performed limited independent quantitative analysis of gene expression.

The validity of the results obtained from arrays is emphasised by the identification in the present study of several known VEGF-induced and angiogenesis-associated genes. COX-2, HB-EGF, decay accelerating factor, and interleukin-8 were previously demonstrated to be up-regulated by VEGF. Stanniocalcin-1, interleukin-8, Nidogen-2, phospholipase A2-γ, and COX-2 were induced in mRNA profiling of capillary formation in collagen matrices, whereas stanniocalcin-1, sprouty, α2-macroglobulin and Egr-1 were upregulated in cDNA microarray analysis of a similar model of capillary morphogenesis. Ten of the VEGF-regulated genes reported here, including COX-2, HB-EGF and α2-macroglobulin, were also found to be increased by VEGF in HUVECs using a cDNA microarray containing 7267 human genes, of which six (Cot, C3G, NR4A1, DSCR1, HERPUD1, DUSP-5 and Egr3), had not previously been reported to be either regulated by VEGF or angiogenesis-associated. The most likely reason for the restricted number of common VEGF-regulated genes in this study and the previous microarray analysis is the use of different arrays, but additional contributory factors may be that the previous study appeared to use only a single set of arrays, and confirmed increased expression of a limited number of genes by quantitative RT-PCR. In this context, it is relevant to note that several genes which showed marked changes in expression in individual oligonucleotide arrays were not reproducibly altered in replicate analyses at the same time-point. In addition, some genes which were strongly induced in triplicate arrays, including C3G, could not be verified by specific RT-PCR, indicating that false positives are a potential problem in interpreting results from oligonucleotide arrays even when replicate analyses are performed. These results emphasize the importance of complementing array methodology with independent, quantitative methods for the evaluation of changes in expression of specific genes. As well as the use of distinct cDNA arrays, the limited overlap the results show with differentially-regulated genes identified during capillary tubulogenesis is most likely due to the involvement of multiple interacting angiogenic factors during capillary formation and the fact that many genes upregulated during angiogenesis reflect long-term changes, whereas many of the most striking changes identified following treatment of HUVECs with VEGF are rapid in onset.

One of the most salient findings from this study is the identification of multiple rapidly-induced genes which encode transcription factors or transcriptional regulators. Three of the genes most strikingly induced by VEGF were the closely-related ‘orphan’ nuclear receptors, Nur77, Nurr1 and Nor1, which comprise the NR4A family of nuclear receptors. These receptors are respectively discussed in Woronicz et al., Nature 1994 Jan. 20, 367(6460):277-81; Zetterstrom et al, Science 1997 Apr. 11, 276(5310):248-50; and Bandoh et al, Mol Cell Endocrinol. 1995, 115:227-30.

Immediate-early expression of Nur77 (NR4A1) is induced by serum, NGF and several other stimuli mainly in neural and neuroendocrine cells. The restricted and non-overlapping phenotypes exhibited by Nur77, Nurr1 and Nor1 knock-out and transgenic mice indicates that these nuclear receptors perform shared and independent biological functions. Nur77-deficient mice exhibit no overt phenotype, but Nur77 is required for apoptosis induced via the T-cell receptor, and transgenic mice expressing either Nur77 or Nor1 display massive apoptosis and a reduction in thymocytes. It is noteworthy that the serine/threonine kinase, Akt, which is strongly activated by VEGF and a major pathway mediating VEGF-dependent cell survival, has been shown to phosphorylate and inactivate Nur77. Though none of these molecules has been reported to be involved in angiogenesis or endothelial function, the findings presented here suggest that Nur77, Nurr1 and Nor1 may function in partially redundant and compensatory functions important for VEGF regulation of transcription.

Several secreted factors and cytokines were also induced, of which sprouty was also induced during capillary morphogenesis.

A number of genes encoding proteins in the clathrin-mediated endocytotic pathway were increased by VEGF, including EHD3, a recently-identified member of the Eps15-homology (EH) domain-containing family of proteins, and the EH domain-binding protein epsin, which associates with Eps15 and together with Eps15 is a component of the clathrin-associated adaptor-related protein complex (AP-2). Another member of the EH domain protein family, EHD1, complexes with the α-adaptin component of AP-2 and has been implicated in internalisation of the IGF-1 receptor. EHD3 itself has been localised to endocytotic vesicles and colocalised with a transferrin-containing recycling compartment. VEGF-induced upregulation of EHD3 may therefore play a specific role in clathrin-mediated endocytotic processes in endothelial cells, possibly involving intracellular VEGF receptor trafficking.

The following description gives details of the Study that has been carried out. If the source is not given, materials used were of the highest available grade.

Study

Abbreviations used are: Egr-3, early growth response gene-3; HUVECs, human umbilical vein endothelial cells; RT-PCR, reverse transcriptase-polymerase chain reaction.

Cell Culture and VEGF Treatment

HUVECs were purchased from TCS CellWorks, Buckinghamshire, UK. Cells were cultured in endothelial basal medium (EBM) supplemented with 10% foetal bovine serum (FBS), 10 ng/ml human epiderma growth factor, 12 μg/ml bovine brain extract, 50 μg/ml gentamycin sulphate (BioWhitakker, Berkshire, UK). For experimental purposes, fully confluent HUVECs at passages 2-3 were pre-incubated in EBM with 0.3% FBS in the absence of other supplements overnight. Cells were subsequently washed to remove residual serum and incubated with VEGF® & D Systems Europe Ltd, Oxon, UK) and washed twice with PBS prior to RNA extraction.

Oligonucleotide Microarrays

Total RNA was extracted from HUVECs treated with 25 ng/ml VEGF for 0, 45 minutes, 90 minutes, 6 hours and 24 hours, using TRIzol® Reagent (Invitrogen, Paisley, UK) according to the manufacturer's instruction. After RNA was further purified using the RNeasy Kit (Qiagen, West Sussex, UK), reverse transcription was carried out using the SuperScript Choice System Kit (Invitrogen) with an oligo-(dT) primer containing T7 RNA polymerase promoter. The double-stranded cDNA was purified with Phase Lock Gel (Eppendorf, Cambridge, UK), phenol/chloroform extraction and ethanol precipitation. In order to generate biotinylated cRNA, in vitro transcription was performed using an Enzo BioArray High Yield RNA transcription Labelling kit (Affymetrix, Santa Clara, Calif.). After cleaning cRNA with RNeasy Kit, 10 μg of fragmented biotinylated-cRNA was hybridised with Affymetrix GeneChips® (U133A array) at 45° C. for 16 hours with constant rotation at 60 rpm. Chips were then washed, and stained with streptavidin-phycoerythrin in an Affymetrix Fluidics Station and scanned by an Agilent GeneArray Laser Scanner at an excitation wavelength of 488 nm. The preparation and hybridisation of cRNA to arrays at each time-point were performed in triplicate from three independent cell cultures using three arrays at each time of VEGF treatment. These arrays were then compared with three control arrays which had been hybridised with each of three RNA preparations from independent untreated control cells.

Data Analysis

Before comparison analysis, data from each chip were normalised and scaled by Microarray Suite Software5.0 (Affymetrix) using the human housekeeping genes β-actin, GAPDH and ISGF-3 (STAT1) as positive controls. Transcript abundance was determined based on the average of the differences between perfect match and mismatch-intensities for each probe family. A detection P value was generated to decide statistically if a transcript was expressed on a chip. For comparison of two chips, the software generated a change P value, a difference call and a signal log ratio. In the present study, nine pairwise cross-comparisons (three control RNA preparations compared with each of three VEGF-treated RNAs) were generated for each time-point. To determine if a transcript was differentially expressed by VEGF treatment, all data were further analysed using Data Mining Tool Software (Affymetrix). Transcripts showing an increase or decrease were scored, a score of 100 indicating a transcript showing the same change in all nine cross-comparisons. Therefore in the present study, a transcript was considered differentially expressed according to the following criteria: 1) a change P value<0.003 (increased expression) or >0.997 (decreased expression); 2) a score greater than 88.9 (8 out of 9 cross-comparisons showing the same call); 3) an average signal log ratio in nine cross-comparisons of >1 (indicating >2 fold increase) or <−1 (>2 fold decrease).

Reverse Transcription and Real-Time PCR

Total RNA was extracted by using RNeasy Kit (Qiagen) and treated with DNAse I (Qiagen) for 15 min at room temperature. Single strand cDNA was synthesised from 2 μg of total RNA with oligo (dT)₁₂₋₁₈ primer in a 20 μl reaction volume using SuperScript First-Strand Synthesis System for RT-PCR (Invitrogen), and diluted into 200 μl for real-time PCR. Forward and reverse primers for real-time PCR were designed by Primer3 software (http://www-genome.wi.mit.edu/cgi-bin/primer/primer3_www.cgi) using the sequence at the 3′ untranslated region of the gene to ensure the specificity of the fragment, and ensuring that amplified fragments were 200-250 base pairs in size. Primers were synthesised by Sigma Genosys and their ability to specifically amplify fragments of the predicted size was verified by conventional RT-PCR methodology. Real-time PCR was performed by using the LightCycler-FastStart DNA Master SYBR Green I Kit and Lightcycler System (Roche Diagnostics, East Sussex, UK) according to the manufacturer's protocol. A melting curve was used to identify a temperature where only amplicon, and not primer dimers accounted for SYBR green-bound fluorescence. PCR was conducted in duplicate for each sample and RNA preparations from two independent experiments were used for real-time RT-PCR. Data were normalised to the reference gene glyceraldehyde 3-phosphate dehydrogenase (GAPDH), and are presented as the mean fold increase or decrease (±standard deviation) compared with control.

Results

To investigate VEGF-regulated gene expression, cRNA was prepared from confluent cultures of HUVECs that had been treated with VEGF, and hybridised with Affymetrix high-density oligonucleotide arrays representing more than 15,000 human genes. The results, presented as the fold change in expression induced by VEGF calculated from the log of the signal ratio between VEGF-treated and control cells, showed a greater than 2-fold VEGF-induced upregulation of 88 different genes, and a greater than 2-fold down-regulation of 100 genes. Distinct patterns of gene expression were observed at different time-points. After 45 minutes, 18 genes were increased >2-fold in VEGF-treated HUVECs compared with untreated controls, of which 16 were also induced after 90 minutes, and only 1 was upregulated at later times. Tissue Factor was the most strongly induced gene at 45 minutes but was not upregulated at later times. In contrast, 20 genes showing no change at 45 minutes were increased >2-fold after 90 minutes, 28 genes were increased only after 6 hours, and a further 21 only after 24 hours. No significant gene down-regulation by VEGF occurred after 45 minutes, 1 gene was down-regulated after 90 minutes, while 17 were down-regulated after 6 hours, and 82 after 24 hours.

Further analysis of VEGF-regulated expression of selected genes was performed by real-time quantitative RT-PCR at different time-points using GAPDH as a reference gene expression of which was not significantly affected by VEGF at all the time-points examined. The appropriateness of GAPDH as a control for real-time PCR analysis of gene expression was further examined by measuring GAPDH levels in time-matched control cells not treated with VEGF. GAPDH expression in control untreated cells did not change significantly over 48 hours. Real-time RT-PCR was used to examine expression of a known VEGF-regulated gene, COX-2, which oligonucleotide array analysis indicated was induced 3-4-fold at 45 and 90 minutes. COX-2 was markedly induced after a 20 minute treatment with VEGF and this effect was sustained after 45 minutes, thereafter decreasing, but remaining elevated >2-fold above the control level for up to 6 hours.

Transcription factors or factors linked to transcriptional regulation made up the largest functional group of VEGF-induced genes and several transcription factors were among the genes most strongly upregulated by VEGF. In particular, at 45 and 90 minutes, VEGF markedly induced expression of genes encoding the three related orphan nuclear receptors, NR4A1, NR4A2 and NR4A3, also known as Nur77, Nurr 1 and Nor 1. Quantitative RT-PCR analysis of these genes confirmed the results obtained from oligonucleotide array analysis, and showed that maximum expression of these genes occurred at approximately 45 minutes, after which expression declined rapidly, reaching control levels after 3 hours. VEGF increased expression of several other transcription factor genes by >3-fold at 45 minutes including Egr2, cyclic AMP-responsive element modulator type 2, and a human homologue of a drosophila homeobox gene. In addition, after 90 minutes, VEGF induced expression of Egr1 binding protein 2, activating transcription factor 3, the forkhead factor FKHL7 and myocyte enhancer factor 2C. VEGF increased expression at the 6 hour time-point of histone deacetylase 9, a member of the family of class II histone deacetylases which play a key role in transcriptional regulation by opposing the effects of histone acetyltransferases and causing transcriptional repression. Real-time RT-PCR showed that increased HDAC9 expression was detectable after 3 hours, reached a maximum after 6-12 hours, and decreased after 24 hours though remaining above the basal level of expression.

VEGF induced the expression of genes for several cytokines and growth factors after 45 minutes including interleukin-8, heparin-binding EGF-like growth factor, GRO-2, and bone morphogenetic protein 2. Other VEGF-induced genes encoding secreted factors after 90 minutes and 6 hours were stanniocalcin-1 and sprouty homologue 4. After 24 hours genes encoding the growth factors IGF-2 and glia maturation factor-α were increased 3-fold by VEGF. However, the most strongly increased secreted factor at 24 hours was α₂-macroglobulin (6-fold induction), a highly-conserved proteinase inhibitor present in human plasma at high concentrations; see Barrett and Starkey, Biochem J. 1973, 133:709-24. Olsen et al, Proc Natl Acad Sci USA 1996, 93:1792-6, discusses that Stanniocalcin-1 is upregulated during capillary morphogenesis, though its function in angiogenesis or endothelial function is unclear. Further analysis of expression of this gene by quantitative real-time RT-PCR showed that VEGF caused a rapid induction of stanniocalcin-1 that was detectable after 20 minutes, reached a maximum after 45 minutes, and sustained for up to 3 hours, thereafter declining but remaining significantly above the control level for 24 hours.

Several signaling molecules were induced by VEGF, of which the serine/threonine kinase Cot (mitogen-activated protein kinase kinase kinase 8), and the dual specificity phosphatases DUSP-1 (MAP kinase phosphatase 1) and DUSP-5 (also called VH3), were the most prominently expressed at 90 minutes, while the Crk-specific guanine nucleotide-releasing factor C3G (approximately 10-fold induction) was the most markedly induced after 6 hours, and Rac-3 and the cytosolic Ca²⁺-independent phospholipase, phospholipase A₂-α, the most prominent after 24 hours. RT-PCR analysis showed that VEGF-induced Cot mRNA expression was maximal after 45 minutes, remained increased after 90 minutes and decreased to the basal level after 6 h.

VEGF increased expression of two ion channels: the inwardly-rectifying potassium K⁺ channel, Kir 2.1, and the small-conductance Cat²⁺-activated K⁺ channel, SK2 or KCNN2. Analysis of the time-course of SK2 expression by real-time RT-PCR showed that it was induced about 6-fold above the control level after 90 minutes, thereafter declining but remaining significantly above the basal level for up to 48 hours. VEGF also induced expression of genes for two transporter molecules after 6 hours: the Na⁺/K⁺/Cl⁻ cotransporter (SLC12A2 or NKCC1) and the y⁺ system cationic amino acid transporter (CAT1 or SLC7A1). Further analysis of VEGF-induced CAT1 expression showed that it increased 2,5-fold after 45 minutes, remained elevated 2-fold or greater above the control level for up to 6 hours, and returned to the basal level after 24 hours.

Several genes were induced which have a known or putative role in clathrin-mediated endocytosis. The earliest and most strongly-induced gene in this functional category was that encoding the Eps15-homology (EH) domain-containing protein, EHD3. EHD3 was prominently induced after 6 and 24 hours, and this finding was confirmed by quantitative RT-PCR analysis which showed a 6-fold increase after 6 hours and 4-fold induction after 24 hours. VEGF also induced expression at 6 hours of epsin, an EH domain-binding protein that associates with both the clathrin-coated vesicle component, Eps 15, and the clathrin-associated adaptor-related complex 2. Increased expression of the sigma 1 subunit of the clathrin-associated adaptor-related complex 1 also occurred after 24 h.

VEGF markedly induced expression of several known genes of poorly defined function. The most markedly induced gene in this category was DSCR1. DSCR1 expression was increased ˜40-fold after 45 minutes, subsequently declining slowly, but remaining more than 2-fold above the basal level for up to 6 hours. The gene encoding cleft lip and palate associated transmembrane protein 1 (CLPTM1) was also induced by VEGF. The CLPTM1 gene is disrupted by a chromosomal translocation associated with the common birth defect, cleft lip and palate, and has strong homology to two Caenorhabditis elegans genes, but is of otherwise unknown function.

Oligonucleotide array analysis revealed significant down-regulation of several genes after 6 and 24 hours. Several down-regulated genes at 6 hours encode either cell surface proteins or proteins associated with cell-cell junctions, including the tight junction component claudin 5, the gap junction protein connexin 37, epithelial V-like antigen 1 (EVA-1) and the water channel protein, aquaporin 1. VEGF also significantly decreased expression of the TNF ligand superfamily member, TRAIL and 1,2-α-mannosidase, a Golgi-associated enzyme which hydrolyses mannose residues during the processing of mannose-rich oligosaccharide intermediates. Specific RT-PCR analysis of selected down-regulated genes indicated that a significant decrease in expression of connexin 37 was detectable as early as 90 minutes and persisted for up to 24 hours, whereas expression of claudin 5, EVA-1,1,2-α-mannosidase, and TRAIL was decreased at 6 hours and/or earlier times but returned to control levels or above after 12 or 24 hours.

The largest functional class of down-regulated genes after 24 hours treatment with VEGF was cell cycle regulators and other genes associated with mitotic apparatus and DNA synthesis. Genes down-regulated by VEGF at 24 hours also included the largest number of genes of unknown function or identity.

1. A product for therapeutic use, which comprises a gene or the product of a gene selected from:
Histone deacetylase 9;
NR4A1;
NR4A2;
NR4A3;
KCNN2;
EHD3;
ADAMTS-1;
FosB;
CLPTM1;
DUSP-1 (MKP-1);
Solute carrier family 7, member 1 (y+arginine transporter);
Solute carrier family 12 (sodium/potassium/chloride) transporter), member 2;
Vinexin-beta;
GRO-2;
Kir2.1 (inwardly rectifying potassium channel);
EH domain-binding mitotic phosphoprotein (Epsin);
Forhead box C1 (FKHL7/FOXC1);
NGF1-A binding protein 2 (Egr1 binding protein 2);
Activating transcription factor 3;
H2.0-like homeo box 1;
Hypothetical protein DKFZp434F0318;
KIAA1718 protein;
Basic transcription element binding protein 1;
Bone morphogenetic protein; 1
Sprouty (drosophila) homolog 4;
cAMP responsive element modulator;
DnaJ (Hsp40) homolog, subfamily B, member 9; or
Fusion derived from t(12;16) malignant liposarcoma.

2. The product according to claim 1, wherein the gene is an orphan nuclear receptor gene selected from the group consisting of NR4A1, NR4A2 and NR4A3.

3. (canceled)

4. A method for providing arterioprotection or promoting angiogenesis in a person or animal, said method comprising administering an effective amount of a product according to claim 1 to a person or animal in need thereof.

5. The method according to claim 4, wherein the product is a gene or the product of a gene selected from the group consisting of NR4A1, NR4A2 and NR4A3.
